(19) United States
(12) Patent Application Publication
(10) Pub. No.: US 2018/0093064 A1
(43) Pub. Date: Apr. 5, 2018

(54) GAS CONCENTRATOR APPARATUS

(71) Applicant: Peter Wojtach, Coram, NY (US)

(72) Inventor: Peter Wojtach, Coram, NY (US)

(21) Appl. No.: 15/283,429

(22) Filed: Oct. 2, 2016

(51) Int. Cl. A61M 16/10; A61M 16/08; A61M 16/00

(52) U.S. Cl. CPC A61M 16/101; A61M 16/0875; A61M 2205/3334; A61M 2016/003; A61M 16/0057

(57) ABSTRACT

A gas concentrator apparatus for supplying a gas to a patient and preventing leakage of the gas when a supply tube is removed from the gas concentrator apparatus includes a gas storage tank, a chamber, a piston, a membrane, a spring member, and the supply tube. The chamber is in gaseous communication with a gas storage tank for receiving the gas from the gas storage tank. The chamber comprises a piston configured to move the chamber in a forward direction or a reverse direction. The membrane is positioned at an end of the chamber distal to the gas storage tank. The membrane is actuated by a spring member to seal an entrance of the supply tube for preventing leakage of the gas when the supply tube is removed from the gas concentrator apparatus.

### FIELD OF THE INVENTION

The invention disclosed herein generally relates to medical ventilator systems. More particularly, the invention relates to a gas concentrator apparatus for supplying a gas, for example, oxygen, to a patient.

### BACKGROUND

Individuals undergoing treatment at hospitals sometimes have difficulty breathing based on the nature of the ailment. Moreover, some patients may have a failed lung due to an accident. Such disabilities or conditions require external assistance for inspiration and expiration of breathing gases. Conventionally, medical ventilator systems assist individuals or patients in inspiring oxygen and expiring carbon dioxide. Typically, oxygen is supplied from oxygen tanks to the ventilator via flow meters. Flow meters enable the practitioner or nurse to regulate the proportion of oxygen supplied to the patient. If there is a decrease in the percentage of oxygen supplied to the patient, the flow meter is adjusted to supply more oxygen. Alternately, if the supplied air contains high proportion of oxygen, the supply from the oxygen tank is stopped. Sometimes, while using the flow meters, operators tend to leave the flow meters open even when the requirement has been met. This forces oxygen to flow out at high velocity and empty the oxygen cylinders resulting in waste. Moreover, when transporting patients who are supplied oxygen by the oxygen tanks, negligence in the closing of the flow meters leads to unnecessary waste or emptying of the oxygen tanks. Hence, there is a long felt but unresolved need for an apparatus, which prevents the loss of oxygen when supply of oxygen via flow meters is left open due to operator negligence.

### SUMMARY OF THE INVENTION

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

The invention disclosed herein addresses the above-mentioned need for an apparatus, which prevents the loss of oxygen when supply of oxygen via flow meters is left open due to operator negligence. The gas concentrator apparatus, disclosed herein, comprises a gas storage tank, a chamber, a piston, a membrane, a spring member, and the supply tube. The chamber is in gaseous communication with a gas storage tank for receiving the gas from the gas storage tank. The chamber comprises a piston configured to move the chamber in a forward direction or a reverse direction. The membrane is positioned at an end of the chamber distal to the gas storage tank. The membrane is actuated by a spring member to seal an entrance of the supply tube for preventing leakage of the gas when the supply tube is removed from the gas concentrator apparatus.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and structures disclosed herein. The description of a method step or a structure referenced by a numeral in a drawing is applicable to the description of that method step or structure shown by that same numeral in any subsequent drawing herein.

FIG. 1 exemplarily illustrates a schematic diagram of a gas concentrator apparatus.

### DETAILED DESCRIPTION OF THE INVENTION

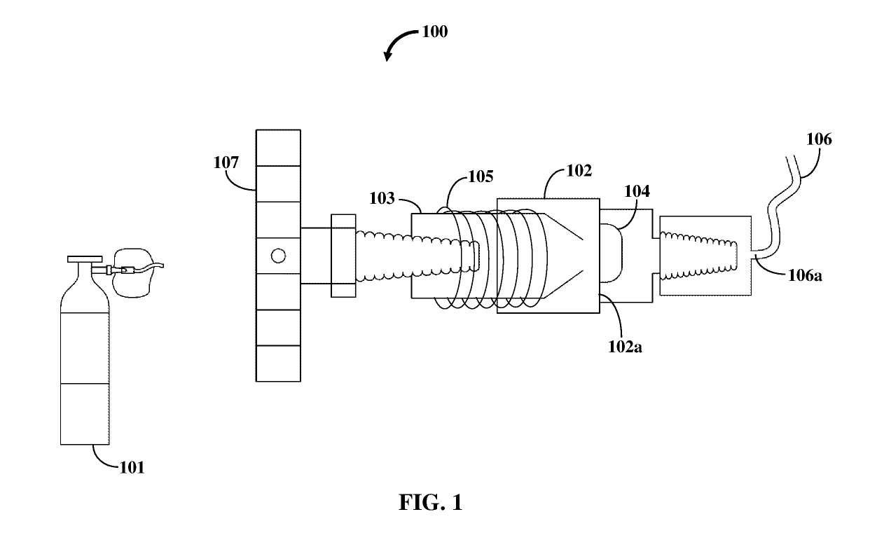

FIG. 1 exemplarily illustrates a schematic diagram of a gas concentrator apparatus 100. The gas concentrator apparatus 100, disclosed herein, comprises a gas storage tank 101, a chamber 102, a piston 103, a membrane 104, a spring member 105, and the supply tube 106. In an embodiment, the chamber 102 is in gaseous communication with the gas storage tank 101 for receiving the gas from the gas storage tank 101. In an embodiment, the gas storage tank 101 is, for example, an oxygen storage tank, etc. The oxygen storage tank is connected to the chamber 102 via the flow meter 107. The flow meter 107 regulates the flow of the gas from the gas storage tank 101 to the chamber 102. The oxygen then flows through the entrance 106a of the supply tube 106 to a patient. In an embodiment, the chamber 102 comprises a piston 103 configured to move the chamber 102 in a forward direction or a reverse direction. The membrane 104 is positioned at an end 102a of the chamber 102 distal to the gas storage tank 101. In an embodiment, the gas concentrator apparatus 100 works to stop the flow of oxygen once the oxygen tubing is disconnected from the gas storage tank 101.

The spring member 105 forces the membrane 104 back over the piston 103, thereby closing the membrane 104 and stopping the air from blowing out of the chamber 102. When there is no use of the oxygen by the patient, the membrane 104 will be in front of the piston 103, how they are by the spring member 105 preventing the flow of oxygen from coming out of the gas storage tank 101. Once the supply tube 106 is manually slid onto the connection end of the gas concentrator apparatus 100, the membrane 104 pushes back against the spring member 105 allowing the oxygen to flow normally from the gas concentrator apparatus 100 at the set rate. The rubber tubing from the supply tube 106 to the patient holds the mechanism in place until the supply tube 106 is removed. In an embodiment, the piston has rubber seals on the end that connects the gas storage tank 101 to securely seal the gas concentrator apparatus 100. In an embodiment, the piston 103 is made of a plastic material. Multiple seals are provided to seal the ends of the movable piston 103 to prevent leakage of the oxygen gas. Moreover, the spring member 105 provides pressure for a rear seal when seated in an oxygen OFF position.

The inner piston 103 has two points where the piston 103 passes through the membrane 104. These two points require seals to prevent escape of the oxygen gas while allowing the mechanism to glide forward and backward. In an embodiment, the membrane 104 is actuated by a spring member 105 to seal an entrance 106a of the supply tube 106 for preventing leakage of the gas when the supply tube 106 is removed from the gas concentrator apparatus 100. The gas concentrator apparatus 100 is a spring-loaded device that prevents the flow of oxygen after the oxygen supply tube 106 is disconnected. The gas concentrator apparatus 100 stops the flow of oxygen and prevents the loss of oxygen from the open flow meter 107. This prevents leakage of oxygen and prolongs the life of the gas storage tank 101. Moreover, the gas concentrator apparatus 100 helps reduce costs by reducing wastage of the oxygen supplied to the patient. In an embodiment, all components of the gas concentrator apparatus 100 will be made of nonmetal materials making the gas concentrator apparatus 100 MRI safe.

The foregoing examples have been provided merely for the purpose of explanation and are in no way to be construed as limiting of the gas concentrator apparatus 100, disclosed herein. While the gas concentrator apparatus 100 has been described with reference to various embodiments, it is understood that the words, which have been used herein, are words of description and illustration, rather than words of limitation. Further, although the gas concentrator apparatus 100, has been described herein with reference to particular means, materials, and embodiments, the gas concentrator apparatus 100 is not intended to be limited to the particulars disclosed herein; rather, the gas concentrator apparatus 100 extends to all functionally equivalent structures, methods and uses, such as are within the scope of the appended claims. Those skilled in the art, having the benefit of the teachings of this specification, may effect numerous modifications thereto and changes may be made without departing from the scope and spirit of the gas concentrator apparatus 100 disclosed herein in their aspects.

What is claimed is:

1. A gas concentrator apparatus for supplying a gas to a patient and preventing leakage of the gas when a supply tube is removed from the gas concentrator apparatus, the gas concentrator apparatus comprising:
a chamber in gaseous communication with a gas storage tank for receiving the gas from the gas storage tank, the chamber comprising a piston configured to move the chamber in one of a forward direction and a reverse direction; and
a membrane positioned at an end of the chamber distal to the gas storage tank, the membrane actuated by a spring member to seal an entrance of the supply tube for preventing leakage of the gas when the supply tube is removed from the gas concentrator apparatus.

2. The gas concentrator apparatus of claim 1, further comprising a flow meter for regulating the flow of the gas from the gas storage tank to the chamber.

3. The gas concentrator apparatus of claim 1, wherein the gas is oxygen.

4. The gas concentrator apparatus of claim 1, wherein the gas storage tank is an oxygen storage tank.
